(19) United States
(12) Patent Application Publication
(10) Pub. No.: US 2008/0065031 A1
(43) Pub. Date: Mar. 13, 2008

(54) COLOSTOMY BAG

(76) Inventor: Ruth Turner, Palm Springs, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/852,084

(22) Filed: Sep. 7, 2007

Related U.S. Application Data
(60) Provisional application No. 60/825,036, filed on Sep. 8, 2006.

(51) Int. Cl. A61F 5/445

(52) U.S. Cl. 604/332

(57) ABSTRACT

A colostomy bag comprises an inner bag and outer bag. The inner bag comprises a sealing device that may be operated before removal from the outer bag to prevent spilling of the contents of the inner bag. A plurality of new inner bags may be stored within the outer bag, and one of the new inner bags may be repositioned inside the outer bag to receive waste from the patient's stoma after an old inner bag has been removed. An air release tube may be coupled to the inner bag to allow air to escape from the inner bag, and the air release tube may be detached from an inner bag that is being discarded and attached to a new inner bag. The new inner bag may be suspended from the outer bag, for example by a hook.

### RELATED APPLICATION

This application claims the benefit of U.S. Provisional Patent Application No. 60/825,036, filed Sep. 8, 2006, which is hereby incorporated by reference in its entirety.

### BACKGROUND

1. Field of the Invention

The invention relates to colostomy bags.

2. Description of the Related Art

A colostomy is a surgical procedure that typically involves connecting a part of a patient's colon onto the anterior abdominal wall, leaving the patient with an opening on the abdomen called a stoma. This opening is generally formed from the end of the large intestine drawn out through the incision and sutured to the skin. After a colostomy, waste from the stomach leaves the patient's body through the stoma, and collects in a bag attached to the patient's abdomen, which is commonly referred to as a colostomy bag. As colostomy patients are all to aware of, existing colostomy bags are often prone to spills due to the design of the bags. In addition, plastic colostomy bags are uncomfortable on the skin of the patient and the bags must be emptied and/or replaced at regular intervals. Because colostomy bags are commonly attached to the patient using adhesives, frequent removal and replacement of colostomy bags tends to irritate the patient's skin. In addition, colostomy bags commonly fill up with air that is received from the stoma and the patient must remove some clothing, such as trousers and underpants, in order to open a portion of the colostomy bag and release the excess air within the bag. Furthermore, whenever the colostomy bag is opened, the risk of spilling the content of the colostomy bag increases. Accordingly, improved colostomy bags that provide better comfort, functionality, and cleanliness are desired.

### SUMMARY

In one embodiment, an apparatus for receiving human waste comprises an outer bag having an access hole and an attachment mechanism for attaching the outer bag to the patient so that the access hole is positioned to receive human waste passed through the stoma and a plurality of inner bags positioned within the outer bag, each inner bag comprising a top end and a bottom end, wherein a bottom end of at least some of the inner bags is releasably coupled to top ends of respective other inner bags, wherein each of the inner bags comprises an access port adapted for coupling to the outer bag such that the access port is positioned to receive human waste passed through the stoma and the access hole of the outer bag, wherein when a particular inner bag is coupled to the outer bag, waste from the stoma flows into the particular inner bag.

In one embodiment, a method for replacing an inner bag of a device configured to receive waste from a patient via a stoma of the patient comprises storing a new inner bag inside an outer bag that is attached to a patient, the new inner bag being configured for receiving waste from a stoma of a patient, opening the outer bag in order to provide access to an old inner bag positioned within the outer bag, the outer bag being configured to allow waste to pass from the patient to the old inner bag via the stoma of the patient, sealing the old inner bag while the old inner bag remains positioned within the outer bag to prevent expulsion of the waste contained in the old inner bag, removing the sealed old inner bag from the outer bag, and positioning the new inner bag inside the outer bag so as to allow the new inner bag to receive waste from the patient via the stoma, the new inner bag being stored within the outer bag while the old inner bag is positioned within the outer bag.

In one embodiment, a kit for receiving biological waste comprises an outer bag having an access hole sized to receive waste from a stoma of a patient, the outer bag having an attachment mechanism for attaching the outer bag to the patient so that the access hole is positioned around the stoma, an inner bag configured for attachment to the outer bag so that an access port of the inner bag is aligned with the access hole of the outer bag, wherein when the inner bag is coupled to the outer bag waste from the stoma flows into the inner bag via an access hole of the inner bag, the inner bag further comprising a device for sealing a portion of the inner bag, and an air release tube for coupling to an upper portion of the inner bag and providing an air release path from the inner bag.

### BRIEF DESCRIPTION OF THE DRAWINGS

FIG. 1A illustrates a colostomy bag that may be attached to a patient.

FIG. 1B illustrates the colostomy bag of FIG. 1A, where an inner bag is placed within the colostomy bag and configured to receive waste from the stoma.

FIGS. 2A-2D each illustrate a colostomy bag having front and rear flaps that are releasably connected so that an inner portion of the colostomy bag may be accessed.

FIG. 3A illustrates a colostomy bag having an inner bag placed within the colostomy bag, and a hole in the inner bag to receive an air release tube.

FIG. 3B illustrates an exemplary air release tube.

FIG. 3C illustrates the colostomy bag of FIG. 3A, where the air release tube is inserted into the hole in the inner bag.

FIG. 3D illustrates the colostomy bag of FIG. 3A, where the air release tube is threaded into the inner bag and the air release tube comprises a mechanism for releasably blocking an air release opening in the tube.

FIG. 4A illustrates a plurality of colostomy bags connected via perforated regions.

FIG. 4B illustrates the plurality of colostomy bags of FIG. 4A, where some of the colostomy bags are unrolled.

FIGS. 5A and 5B illustrate a colostomy bag having a zipper configured to seal a lower portion of the colostomy bag.

FIG. 5C illustrates a colostomy bag having a releasably sealable discharge portion configured to allow empting of the colostomy bag.

FIG. 6A illustrates a colostomy bag having a hook mechanism for supporting one or more inner bags.

FIG. 6B illustrates an inner bag having an opening sized to be supported by the hook mechanism on the bag of FIG. 6A.

FIG. 6C illustrates the inner bag of FIG. 6B within the outer bag of FIG. 6A, the inner bag being supported on the hook mechanism of the outer bag.

FIG. 6D illustrates a colostomy bag having protrusions for supporting one or more inner bags.

FIG. 6E illustrates an inner bag having openings sized to be supported by the protrusions on the bag of FIG. 6D.

FIG. 6F illustrates the inner bag of FIG. 6E within the outer bag of FIG. 6D, the inner bag being supported on the protrusions of the outer bag.

FIGS. 7A and 7B illustrate a colostomy bag having a string configure to seal a lower portion of the bag, such as when the lower portion is filled with waste.

FIG. 8A is a front view of a colostomy bag cover.

FIG. 8B is a rear view of the colostomy bag cover illustrated in FIG. 8A.

FIG. 8C is a front view of another colostomy bag cover comprising an attachment mechanism for closing an opening in the cover.

### DETAILED DESCRIPTION OF CERTAIN EMBODIMENTS

Embodiments of the invention will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

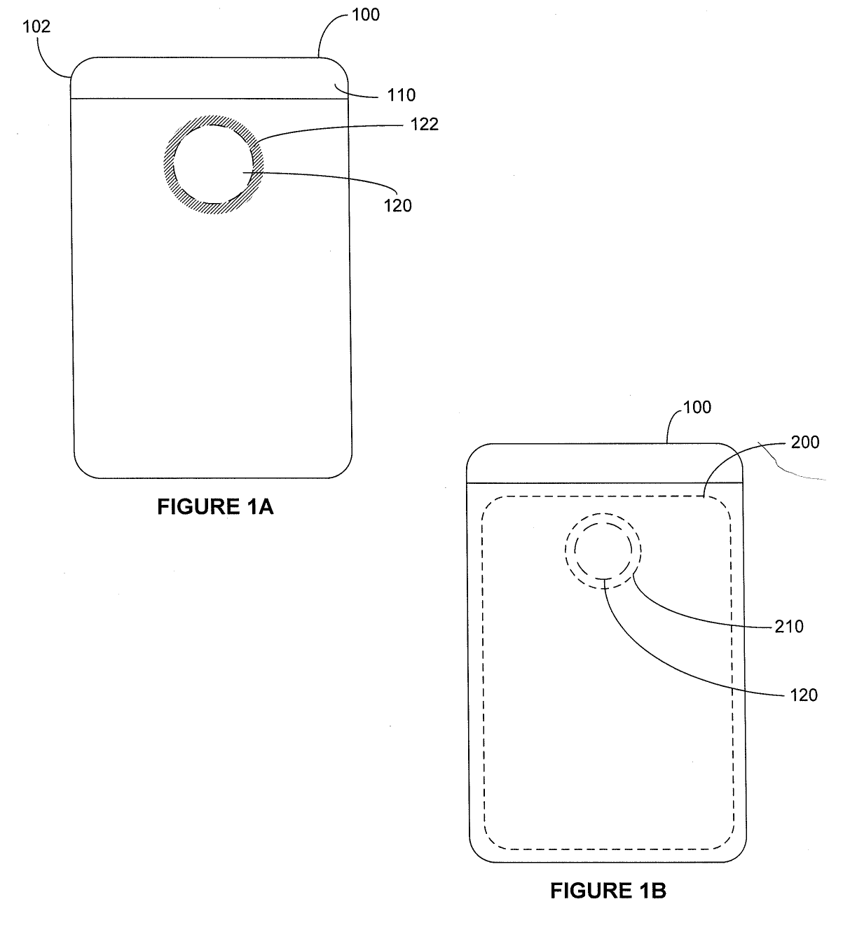

FIG. 1A illustrates a colostomy bag that may be attached to a patient. In the embodiment of FIG. 1A, the colostomy bag 100, also referred to herein as an outer colostomy bag, comprises an access panel 110 that may be opened by lifting the access panel 110 to expose an opening at the top 102 of the colostomy bag 100. Thus, in this embodiment the colostomy bag 100 may be opened in a manner similar to the method used to open the flap on a pillow case. Other mechanisms for providing access to an opening of the colostomy bag 100, some of which are discussed below, are also contemplated.

The exemplary colostomy bag 100 comprises an access hole 120 sized to be placed around an opening on a patient, such as a stoma that results from surgery performed on the patient. In operation, the access hole 120 may have an adhesive 122 surrounding the access hole 120 that adheres to the patient's skin and bonds the colostomy bag 100 to the patient. Thus, the bonding between the adhesive 122 and the patient's skin must be broken in order to remove the colostomy bag 100 and replace the colostomy bag 100 with a new bag.

FIG. 1B illustrates the colostomy bag of FIG. 1A, where an inner bag 200 is placed within the colostomy bag 100 and configured to receive waste from the stoma. In this embodiment, the inner bag 200 comprises an access port 210 that is sized larger than the access hole 120. In one embodiment, the access port 210 is adhered to an inner surface of the colostomy bag 100 so that the access port 210 surrounds the access hole 120 and waste passing through the access hole 120 goes into the inner bag 200. Accordingly, as waste is expelled from the stoma, the waste is accumulated in the inner bag 200, rather than the colostomy bag 100, which is adhered to be patient's skin, such as via adhesive 122 (FIG. 1A). In this embodiment, when the inner bag 200 needs to be replaced, the attachment of the colostomy bag 100 to the patient does not need to be broken, but only the attachment of the inner bag 200 to the colostomy bag 100. Thus, by using the inner bag 200, the patient to which the colostomy bag 100 is attached experiences reduced irritation and/or pain when the inner bag 200 is periodically replaced, as the colostomy bag 100 may remain adhered to the patient while the inner bag 200 is replaced.

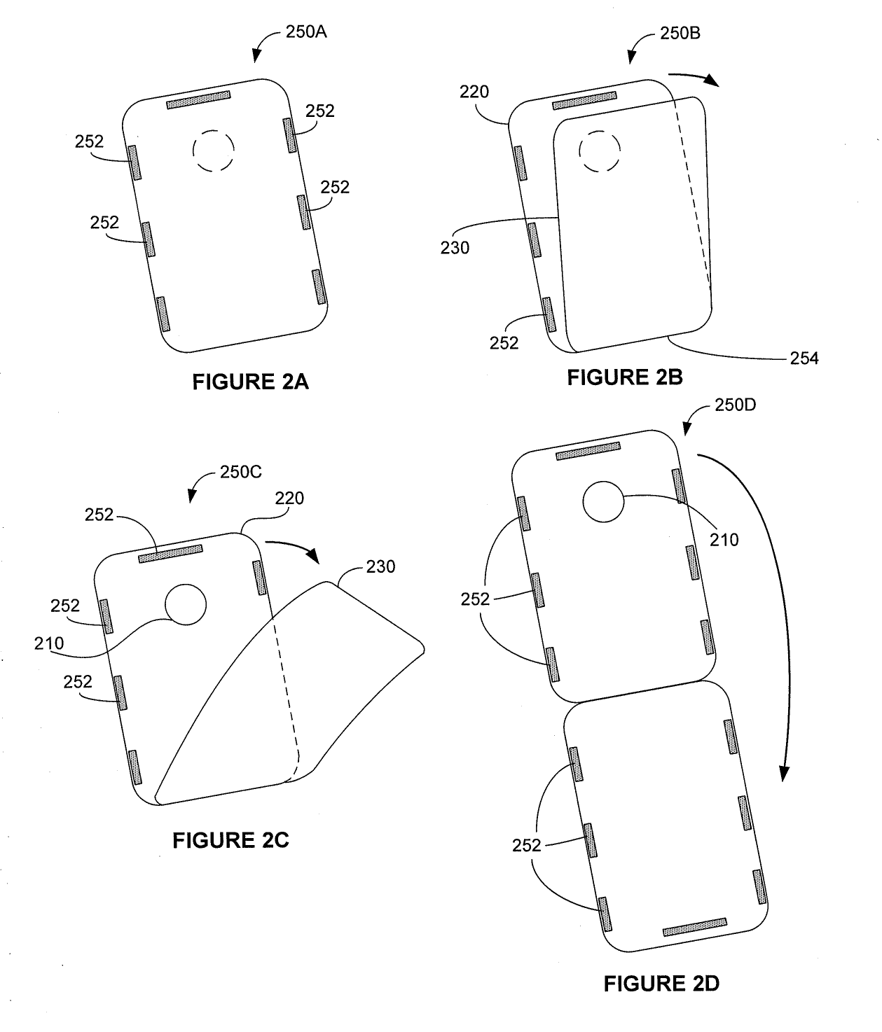

FIGS. 2A-2D each illustrate a colostomy bag 250 having a front flap 230 and rear flap 220 that are releasably connected so that an inner portion of the colostomy bag 250 may be accessed, such as to remove and replace an inner colostomy bag. In the embodiment of FIGS. 2A-2D, the colostomy bag 250 comprises multiple attachment devices 252 that secure the front flap 230 to the rear flap 220. In one embodiment, the attachment devices comprise hook and loop materials, such as Velcro®. In other embodiments, the attachment devices may include any other device, such as snaps, zippers, clips, latches, or buttons, that allow the front flap 230 to be secured to the rear flap 220.

In FIG. 2A, the colostomy bag 250A is in a closed position. In this position, the attachment devices 252 secure the front flap 230 to the rear flap 220. In one embodiment, an inner bag is disposed between the front and rear flaps 230, 220. In FIG. 2B, the front and rear flaps 230, 220 have been separated from one another on three sides so that they are independently moveable. As shown in FIG. 2B, bottom portions 254 of each of the flaps 230, 220 are permanently secured. Thus, the flaps 230, 220 pivot around the bottom portions 254. Moving to FIG. 2C, the front flap 230 has been moved further from the rear flap 220 to expose the access port 210 in the colostomy bag 250. In FIG. 2D, the colostomy bag 250D is completely opened, providing access to the access port 210 of the colostomy bag 250D, and allowing easier attachment of an inner bag around the access port 210.

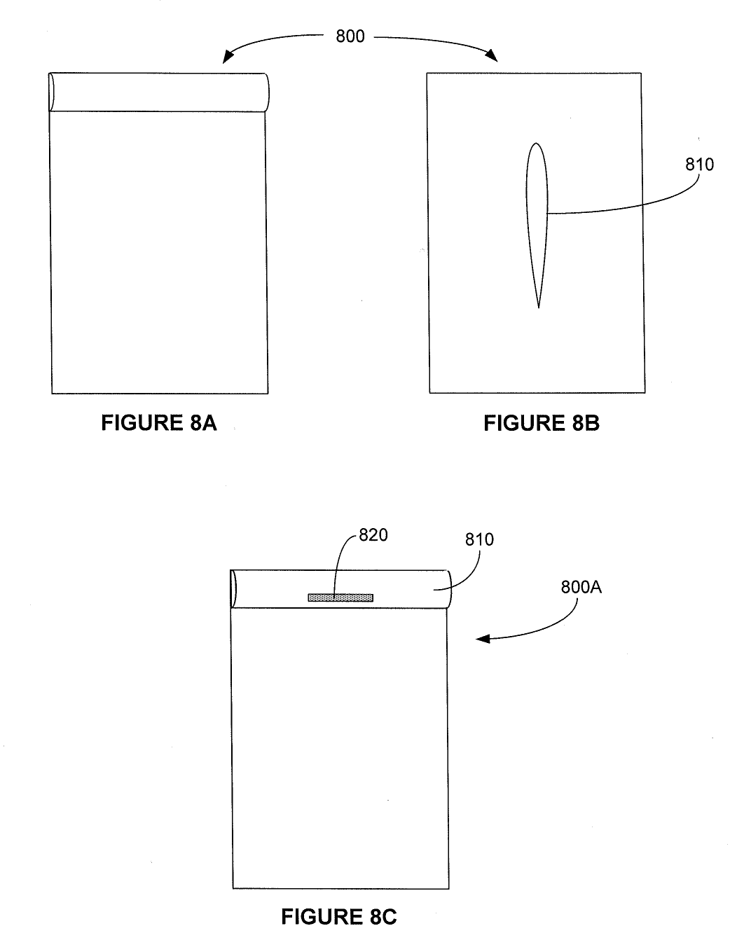

In one embodiment, the colostomy bag 100, 250 may include a soft material, such as flocking, on a surface of the bag that contacts the patient's skin. In this embodiment, irritation to the patient's skin may be reduced. Other materials that reduce skin irritation may also be used to cover portions of the colostomy bag. In another embodiment, such as illustrated in FIGS. 8A-8C, for example, a separate cover may be placed around the colostomy bag to increase the patient's comfort.

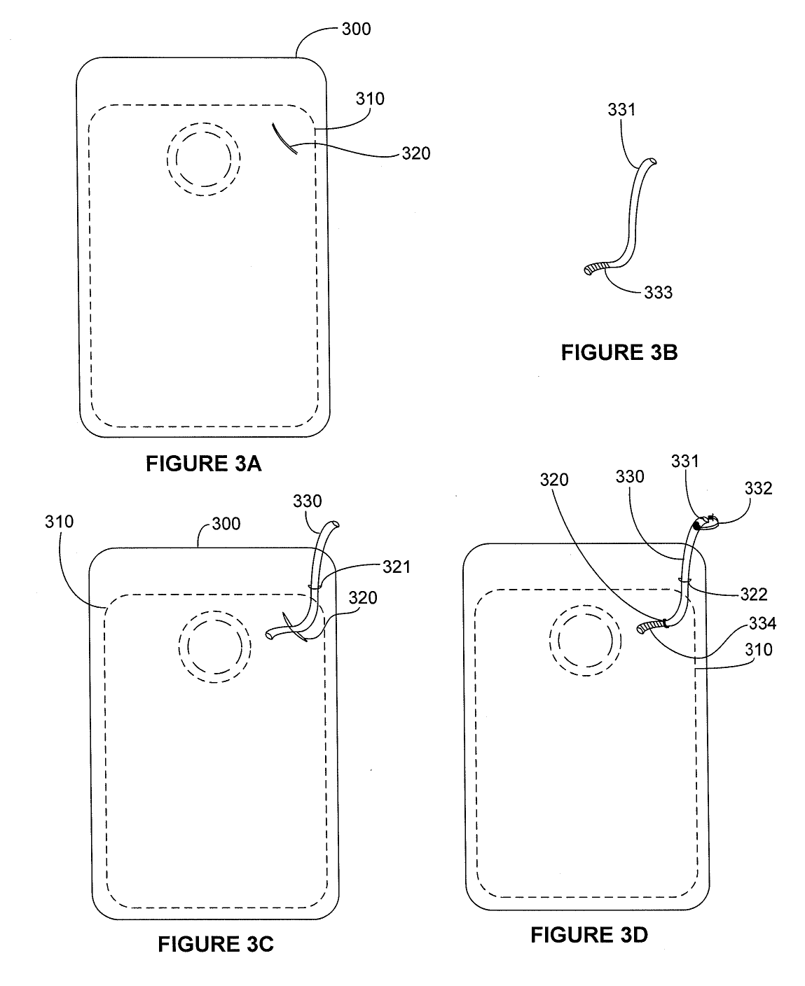

FIG. 3A illustrates a colostomy bag 300 having an inner bag 310 placed within the colostomy bag 300, wherein the inner bag 310 comprises a hole 320 sized to receive an air release tube. In FIG. 3B, an air release tube 330 is illustrated inserted through the hole 320 in the inner bag 310. As those of skill in the art will recognize, gas is periodically expelled from the stoma and may cause colostomy bags to quickly fill with gas, rather than solid waste. In order to reduce the difficulty involved with releasing gas from a colostomy bag, embodiments are described with respect to FIGS. 3C and 3D, for example, having an air release tube inserted into a colostomy bag.

FIG. 3B illustrates an exemplary air release tube 331 comprising a threaded portion 333 that is configured to threadedly engage with a hole in a colostomy bag. The air release tube 331 may be inserted into a colostomy bag, such as an inner colostomy bag inside of an outer colostomy bag, by rotating the air release tube 330 while applying pressure between the threaded portion 333 and a correspondingly sized hole in the colostomy bag.

As shown in FIG. 3C, the air release tube 330, which may comprise any type of tubing, such as flexible rubber tubing, plastic tubing, or metal tubing, is inserted into the inner bag 310 via the hole 320. When the patient, or caregiver to the patient, desires to release gas from the inner bag 310, the air release tube 330 may be inserted into the inner bag 310 via the hole 320, and when pressure is applied to the inner bag 310, gas within the inner bag 310 will exit the air release tube 330. Accordingly, the inner bag 310 may be used for an extended time period due to the evacuation of gas that periodically accumulates within the inner bag 310.

In one embodiment, the air release tube 330 is inserted into the colostomy bag 300 underneath an opening in a top of the bag, which in one embodiment opens similar to the flap on a pillow case. In another embodiment, the air release tube 330 may enter the inside portion of the bag through the sides of the bag, such as between the attachment devices 252 of FIG. 2. In another embodiment, the colostomy bag 300 comprises a hole 321 through which the air release tube 330 may be inserted.

FIG. 3D illustrates the colostomy bag of FIG. 3A, where the air release tube 330 inserted into the inner bag 310 comprises a mechanism for releasably blocking an air release opening 331 in the tube 330. As illustrated in FIG. 3D, the air release tube 330 comprises a blocking mechanism 332 that may be used to block the opening 331 of the air release tube 330 in order to seal the contents of the inner bag 310. In order to reduce the risk of waste from the inner bag 310 escaping inadvertently through the air release tube 330, the blocking mechanism 332, when inserted into the opening 331 of the air release tube 330, seals the air release tube 330 and thereby preventing passage of waste out of the air release tube 330. Other mechanisms for sealing an end of the air release tube 330, including valves, are also contemplated and are usable within the scope of the systems and methods described herein.

In the embodiment of FIG. 3D, the air release tube 330 also comprises a threaded portion 334 that provides a coupling between the air release tube 330 and the hole 320. In this embodiment, the air release tube 330 may be inserted into the inner bag 310 by rotating the air release tube 330 while applying pressure between the threaded portion 334 and a hole 322. The spiral threads on the threaded portion 334 cause the air release tube 330 to enter the inner bag 310 through the hole 322. Advantageously, the threaded portion 334 provides an enhanced engagement of the air release tube 330 and the inner bag 310. In other embodiments, other attachment means, such as protrusions or indentations on the air release tube 330, may be used in order to engage the air release tube 330 to the inner bag 310.

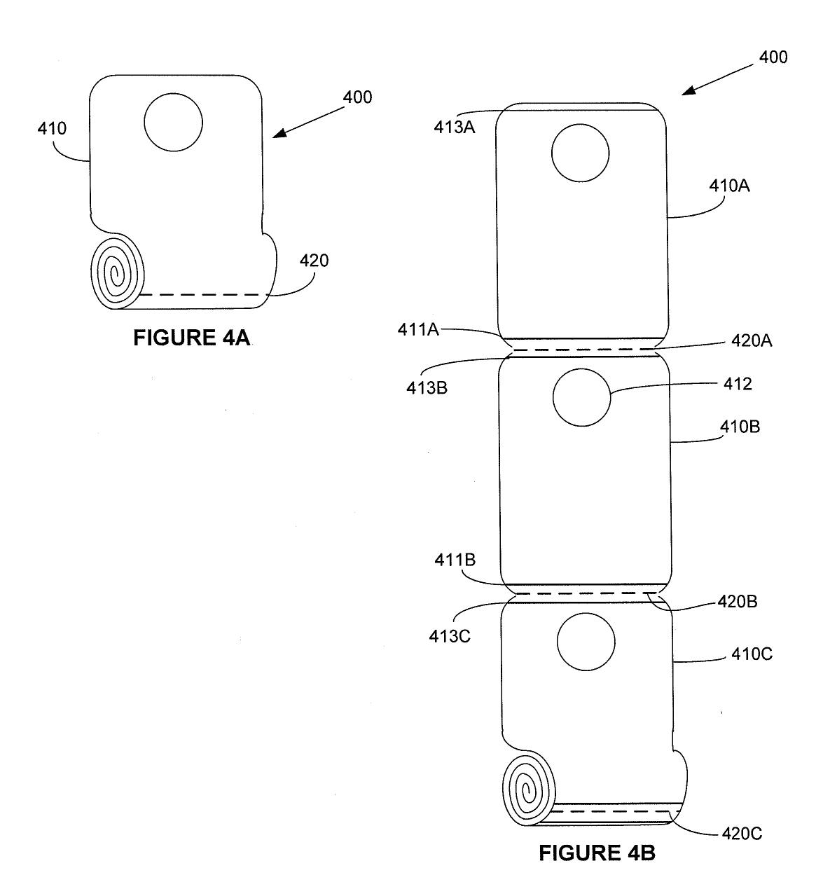

FIGS. 4A and 4B illustrate a plurality 400 of colostomy bags 410 connected via perforated regions 420. As shown in FIGS. 4A and 4B, the colostomy bags 410 may be rolled up, or otherwise compressed, in order to decrease the amount of space required to store the plurality 400 of colostomy bags 410. In this embodiment, a perforated region 420 separates each pair of colostomy bags so that, for example, when colostomy bag 410A is removed, the perforation 420A may be torn, separating the colostomy bags 410A and 410B. Once separated, the colostomy bag 410A may be discarded and the colostomy bag 410B may be positioned to receive waste from a stoma. In this embodiment, multiple colostomy bags may be easily stored so that they are readily available for use. In the embodiment of FIG. 4B, each of the colostomy bags 410 comprise upper sealed portions 413 and lower sealed portions 411 that prevent leakage of waste through respective upper and lower portions of the colostomy bags 410. For example, the lower sealed portion 411A of the colostomy bag 410A prevents leakage out of the bottom of the bag after tearing the perforated region 420A. In one embodiment, the colostomy bags 410 do not have the upper sealed portion 413A.

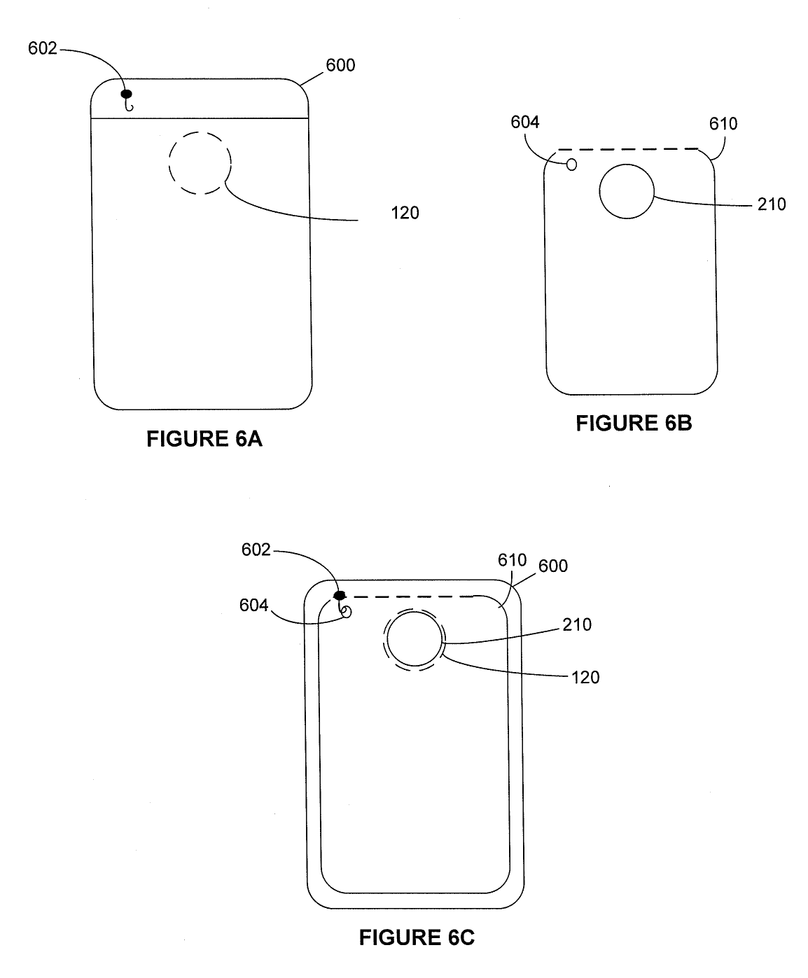

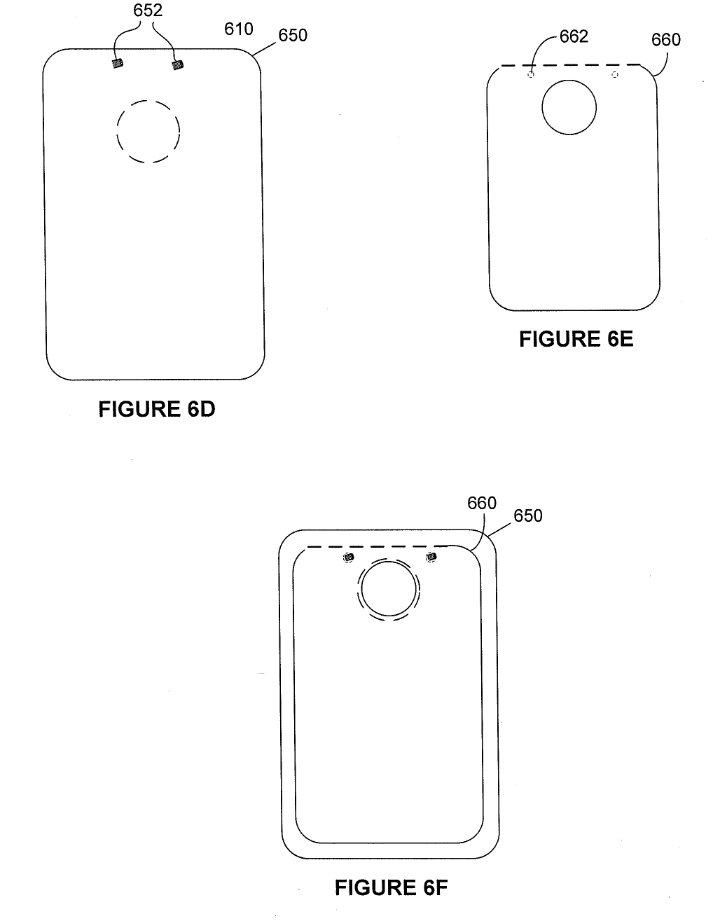

In one embodiment, the inner bags 410 are inner bags configured for placement within an outer colostomy bag, such as outer colostomy bag 100, and used to receive waste through the access hole 120 of the colostomy bag 100. Thus, in one embodiment the plurality 400 of inner bags 410 may be placed within the colostomy bag 100 and sequentially used to receive waste from the stoma. In this embodiment, once a first inner bag, such as inner bag 410A, is ready for removal, the inner bag 410A may be closed and separated from the remaining inner bags 410 via the perforation 420A. After removal of the inner bag 410A, the inner bag 410B may be placed so that an access port 412 of the inner bag 410B surrounds the access hole 120 of the colostomy bag 100. In this way, multiple inner colostomy bags, such as inner bags 410, may be stored and transported inside the colostomy bag 100. In certain embodiments, each of the inner bags 410 comprises an attachment mechanism configured for attachment to an outer bag, such as those described with reference to FIGS. 6A-6F, for example.

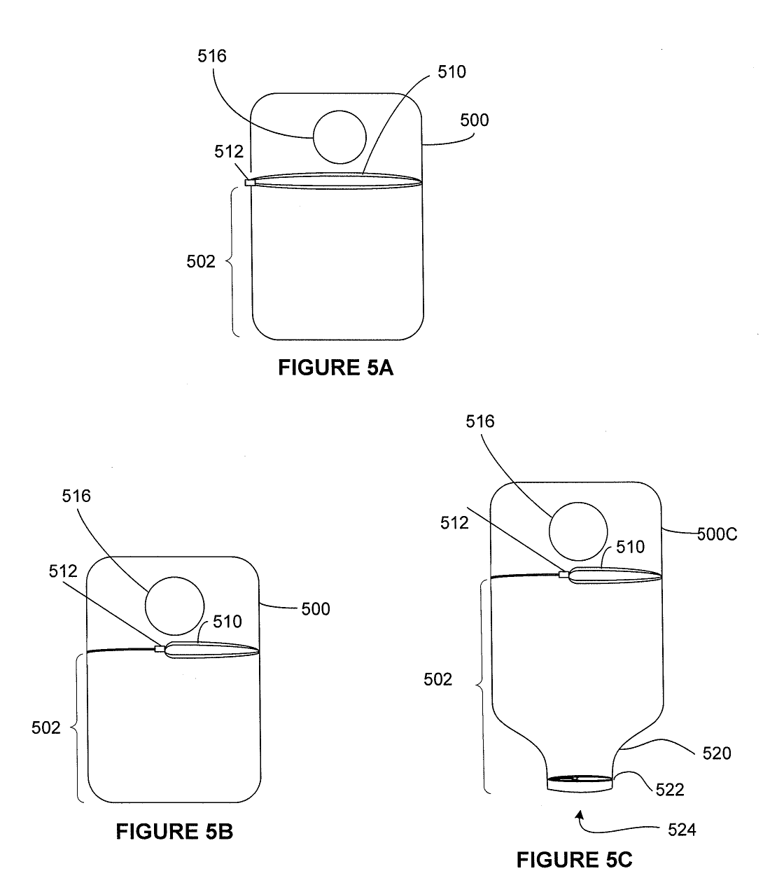

FIGS. 5A and 5B illustrate a colostomy bag 500 having a zipper 510 configure to seal a lower portion 502 of the bag, such as when the lower portion 502 is filled with waste. As shown in FIGS. 5A and 5B, the zipper 510 comprises a lever 512 that is movable from one side of the zipper 510 to the other side of the zipper 510, whereby the lever 512 causes the opposing sides of the zipper 510 to interlock, sealing the lower portion 502 of the colostomy bag 500 so that the content of the bag cannot escape through the access port 516 of the bag 500. In the embodiment of FIG. 5A, the bag 500 is shown with the zipper 510 in an open position, such that waste that enters through the access port 516 will enter the lower portion 502 of the bag 500. In the embodiment of FIG. 5B, the zipper 510 is partially closed. When the zipper 510 is moved to the right side of the bag 500, as illustrated in FIG. 5B, the lower portion 502 is sealed off from the access port 516.

FIG. 5C illustrates a colostomy bag 500C having a releasably sealable discharge portion 520 configured to allow empting of the colostomy bag 500C. Exemplary colostomy bag 500C comprises a sealing mechanism 522, such as a plastic zipper, that is configured to allow the content of the colostomy bag 500C to be emptied through a lower discharge opening 524 of the colostomy bag 500C. The sealing mechanism 522 is further configured to seal the bag 500C so that the content of the bag 500C cannot escape through the discharge opening 524, such as after the content of the bag 500C are emptied into a toilet, bucket, or tub, for example. In this way, exemplary colostomy bag 500C is reusable, allowing a patient to empty the bag 500C, which may be a colostomy bag attached directly to the patient or an inner colostomy bag inside an outer colostomy bag, for example, without removing the bag 500C. Depending on the embodiment, the discharge portion 520 may be shaped differently than illustrated in FIG. 5C and/or the sealing mechanism 522 may comprise any suitable mechanism that releasably opens and seals the discharge portion 520.

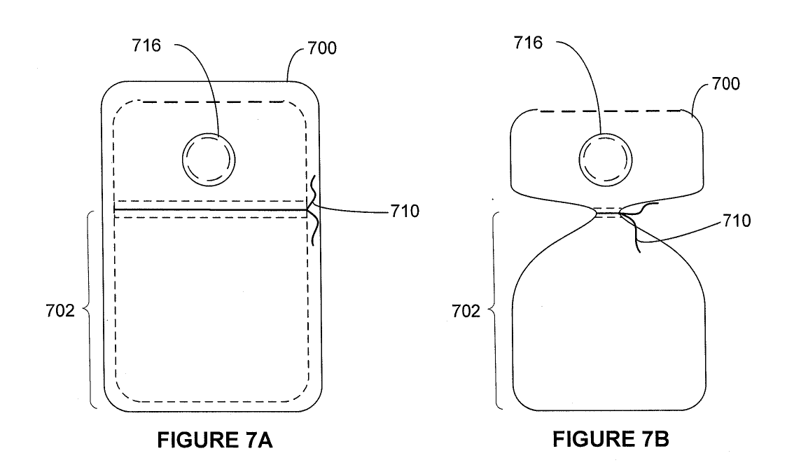

FIGS. 7A and 7B illustrate an outer colostomy bag 700 and an inner bag 704 having a string 710 configure to seal a lower portion 702 of the inner bag 704, such as when the lower portion 702 is filled with waste. As shown in FIGS. 7A and 7B, the string 710 is threaded around a circumference of the inner bag 704 below the access port 516. In order to seal the lower portion 702 of the inner bag 704, the string may be pulled tight, thereby cinching in a portion of the inner bag 704 and created a seal about the lower portion 702. In the embodiment of FIG. 7A, the inner bag 704 is shown with the string 710 relaxed, such that waste that enters through the access port 716 will enter the lower portion 702 of the inner bag 704. In the embodiment of FIG. 7B, the string 710 has been pulled, such that the lower portion 502 is substantially sealed off from the access port 516. In other embodiments, a wire or zip tie, for example, may be used in place of the string 710. In some embodiments, the string 710 is not threaded around the inner bag 704, but may be manually placed around the bag 704 immediately prior to sealing the lower portion 702.

In one embodiment, the colostomy bags 500 and 700 each comprise an inner bag, such as those that are configured for placement inside the colostomy bag 100 for coupling around the access hole 120 to receive waste from a stoma. In some embodiments, the colostomy bag 100 also comprises a zipper, such as the zipper 510, or other sealing means, which provides access to an inner portion of the colostomy bag 100.

FIG. 6A illustrates an outer colostomy bag 600 having a hook mechanism 602 for supporting one or more inner bags within the outer colostomy bag 600. FIG. 6B illustrates an inner bag 610 having an opening 604 sized to be supported by the hook mechanism 602 on the bag of FIG. 6A. FIG. 6C illustrates the outer colostomy bag 600 of FIG. 6A having the inner bag 610 of FIG. 6B within the outer colostomy bag 600 and supported on the hook mechanism 602 on the outer colostomy bag 600. In the embodiments of FIGS. 6A, 6B, and 6C, the inner bag 610 is supported within the outer colostomy bag 600 via engagement of the hook mechanism 602 with the opening 604. With the attachment of the inner bag 610 with the outer colostomy bag 600 in this manner, the pressure of the waste within the inner bag 610 is not entirely supported by the adhesive attachment of the access port 210 around the access hole 120.

FIG. 6D illustrates a colostomy bag 650 having protrusions 652 for supporting one or more inner bags within the colostomy bag 650. FIG. 6E illustrates an inner bag 660 having openings 662 sized to be supported by the protrusions 652 on the bag 650 of FIG. 6D. FIG. 6E illustrates the outer bag 650 of FIG. 6D having the inner bag 660 of FIG. 6E within the outer bag 650 and supported on the protrusions 652 on the outer bag 650. In the embodiment of FIG. 6F, the inner bag 660 is supported within the outer bag 650 via engagement of the protrusions 652 with the openings 662. With the attachment of the inner bag 660 within the outer bag 650 in this manner, the pressure of the waste within the inner bag 660 is not entirely supported by the adhesive attachment of the access port (inner bag) around the access hole (outer bag). In one embodiment, the inner bag 660 comprises perforated regions that are used to create the holes 662. For example, when the protrusions 652 are pressed against the perforated regions, a portion of the inner bag 660 surrounded by the perforated regions are at least partially displaced from connection with the inner bag 660 in order to form the holes 662 through which the protrusion 652 extend. In the embodiment of FIGS. 6D and 6F, the protrusions are shown as cylindrical posts; however, any other suitably shaped protrusion may be used also. In other embodiments, alternative attachment mechanisms may be used to secure the inner bags 660 to the colostomy bag 650. For example, in one embodiment hook and loop attachment means, snaps, or buttons, are used to secure the inner bag 660 to the colostomy bag 650.

FIG. 8A is a front view of a colostomy bag cover 800. In this embodiment, the colostomy bag cover 800 may be placed around a colostomy bag. In the embodiment of FIG. 8A-8C, the colostomy bag cover 800 may be opened in a manner similar to the method used to open the flap on a pillow case.

FIG. 8B is a rear view of the colostomy bag cover 800 illustrated in FIG. 8A. As illustrated in FIG. 8B, the colostomy bag cover 800 comprises an opening 805 sized to fit around the access port of a colostomy bag, such as the access port 210 (FIG. 2). Thus, the colostomy bag cover 800 can remain around a colostomy bag when the colostomy bag is attached to a patient, thereby providing a barrier between the colostomy bag and the patient's skin. In one embodiment, at least the rear surface of the colostomy bag cover 800 that contacts the patient's skin comprises a soft material, such as flocking. In this embodiment, irritation to the patient's skin may be reduced.

FIG. 8C is a front view of another colostomy bag cover 800A comprising an attachment mechanism 820 for closing an opening in the cover 800A. In one embodiment, the attachment mechanism comprises hook and loop attachment materials on a flap 810 of the bag 800A. In this embodiment, the flap 810 of the cover 800A may be secured to the main portion of the cover 800A via strips of hook and loop attachment materials, such as Velcro®. Thus, in one embodiment the underside of the flap 810 comprises a strip of hook (or loop) material, while the outside of the cover 800A comprises a corresponding strip of loop (or hook) material that interlocks and seals the cover 800A. In one embodiment, the inner bags, such as inner bags 410 also comprise hook and loop attachment mechanisms that seal the bags. In one embodiment, the only opening in the cover 800 is the opening 805, wherein the cover 800 is positioned around the colostomy bag by inserting the colostomy bag into the cover through the opening 805.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. An apparatus for receiving human waste, the apparatus comprising:
an outer bag having an access hole and an attachment mechanism for attaching the outer bag to the patient so that the access hole is positioned to receive human waste passed through the stoma; and
a plurality of inner bags positioned within the outer bag, each inner bag comprising a top end and a bottom end, wherein a bottom end of at least some of the inner bags is releasably coupled to top ends of respective other inner bags, wherein each of the inner bags comprises an access port adapted for coupling to the outer bag such that the access port is positioned to receive human waste passed through the stoma and the access hole of the outer bag, wherein when a particular inner bag is coupled to the outer bag, waste from the stoma flows into the particular inner bag.

2. The colostomy bag of claim 1, wherein at least one of the inner bags comprises an opening in an upper portion of the inner bag, the opening sized to receive an air release tube.

3. The colostomy bag of claim 2, wherein the air release tube comprises protrusions that are passed through the opening of the inner bag so that a portion of the air release tube is stabilized within the inner bag.

4. The colostomy bag of claim 1, wherein at least one of the inner bags comprises an opening adapted for attachment to a securing mechanism coupled to the outer bag.

5. The colostomy bag of claim 4, wherein the securing mechanism comprises a hook.

6. A method for replacing an inner bag of a device configured to receive waste from a patient via a stoma of the patient, the method comprising:
storing a new inner bag inside an outer bag that is attached to a patient, the new inner bag being configured for receiving waste from a stoma of a patient;
opening the outer bag in order to provide access to an old inner bag positioned within the outer bag, the outer bag being configured to allow waste to pass from the patient to the old inner bag via the stoma of the patient;
sealing the old inner bag while the old inner bag remains positioned within the outer bag to prevent expulsion of the waste contained in the old inner bag;
removing the sealed old inner bag from the outer bag; and
positioning the new inner bag inside the outer bag so as to allow the new inner bag to receive waste from the patient via the stoma, the new inner bag being stored within the outer bag while the old inner bag is positioned within the outer bag.

7. The method of claim 6, further comprising detaching the new inner bag from a plurality of new inner bags positioned inside the outer bag.

8. The method of claim 6, wherein the old inner bag and the new inner bag each comprise an access port configured to receive waste from the patient.

9. The method of claim 8, wherein the old inner bag is sealed below the access port using a zipping device.

10. The method of claim 8, wherein the old inner bag is sealed below the access port by cinching the old inner bag around a circumference of the old inner bag.

11. The method of claim 10, wherein the old inner bag is cinched around the circumference using one or more of a wire, a string, a thread, a rope, a zip tie, and a strand of another flexible material.

12. The method of claim 6, further comprising uncoupling an air release tube from the old inner bag, the air release tube being configured to allow air to escape from the old inner bag.

13. The method of claim 12, further comprising removing the air release tube from the old inner bag and coupling the air release tube to the new inner bag.

14. The method of claim 13, wherein the air release tube comprises a threaded portion configured to engage an opening on the new inner bag.

15. The method of claim 6, further comprising suspending the inner bag from the outer bag by coupling the inner bag to a securing mechanism coupled to the outer bag at a location above the access port of the inner bag.

16. The method of claim 6, wherein the outer bag remains coupled to the patient during the steps of storing, opening, sealing, removing and positioning.

17. A kit for receiving biological waste, the kit comprising:
an outer bag having an access hole sized to receive waste from a stoma of a patient, the outer bag having an attachment mechanism for attaching the outer bag to the patient so that the access hole is positioned around the stoma;
an inner bag configured for attachment to the outer bag so that an access port of the inner bag is aligned with the access hole of the outer bag, wherein when the inner bag is coupled to the outer bag waste from the stoma flows into the inner bag via an access hole of the inner bag, the inner bag further comprising a device for sealing a portion of the inner bag; and
an air release tube for coupling to an upper portion of the inner bag and providing an air release path from the inner bag.

18. The kit of claim 17, wherein the inner bag is configured to be sealed by a zipper mechanism.

19. The kit of claim 17, wherein the air release tube is removably coupled to the inner bag.

20. The kit of claim 17, comprising a plurality of inner bags each comprising a top end and a bottom end, wherein a bottom end of at least some of the inner bags is coupled to top ends of respective other inner bags
